(19) United States
(12) Patent Application Publication
(10) Pub. No.: US 2017/0218522 A1
(43) Pub. Date: Aug. 3, 2017

(54) METALLIC WORKPIECE OF TITANIUM AND/OR A TITANIUM ALLOY AND/OR NICKEL-TITANIUM ALLOYS AND ALSO NITINOL WITH A POROUS SURFACE AND PRODUCTION PROCESS

(71) Applicant: Christian-Albrechts-Universitaet zu Kiel, Kiel (DE)

(72) Inventors: Melike Baytekin-Gerngross, Kiel (DE); Mark-Daniel Gerngross, Kiel (DE); Rainer Adelung, Kiel (DE); Juergen Carstensen, Kiel (DE)

(21) Appl. No.: 15/328,939

(22) PCT Filed: Jul. 31, 2015
(86) PCT No.: PCT/DE2015/100322

§ 371 Date: Jan. 25, 2017

(30) Foreign Application Priority Data
Jul. 31, 2014 (DE) 10 2014 110 922.8

(51) Int. Cl. C23F 1/26; A61L 27/56; A61L 27/06; C23F 1/30; C22C 14/00

(52) U.S. Cl. CPC C23F 1/26; C23F 1/30; C22C 14/00; A61L 27/06; A61L 27/56; A61L 2430/02; A61L 2400/18

(57) ABSTRACT

A surface-treated metallic workpiece of titanium and/or titanium alloys with titanium as the main constituent and/or nickel-titanium alloys and also nitinol, wherein on the treated surface the metal is free from inclusions, precipitates of other metals, accumulations of alkali metals, alkaline earth metals and/or aluminium, intermetallic phases, and/or mechanically highly defect-rich regions, and the surface has a first roughness and a second roughness, wherein the first roughness is provided by depressions in the form of pores, the pores having a diameter in the range between 0.5 and 50 μm—being open in the direction of the surface and closed in the direction of the workpiece, and at least some of the pores having an undercut, and the second roughness is provided by randomly distributed elevations and depressions in the range of 100 nm and less. The invention also relates to a production process for a surface-treated workpiece.

The present invention relates to the field of producing surfaces on metallic workpieces with improved adhesion to organic polymers and/or biological materials and/or ceramic materials.

The invention relates to a surface-treated metallic workpiece of titanium and/or titanium alloys with titanium as a main component and/or nickel-titanium alloys as well as Nitinol, wherein the treated surface of the metal is free from inclusions, precipitations of other metals, alkaline earth metals and/or aluminum, intermetallic phases, and/or mechanically intensely defect-rich areas, and the surface has a first roughness and a second roughness, wherein the first roughness is provided by depressions in the form of pores, the pores having a diameter in the range between 0.5 and 50 μm, are open towards the surface, and are closed towards the workpiece, and at least a portion of the pores have an undercut, and the second roughness is provided by statistically distributed elevations and depressions in the range of 100 nm and less.

Furthermore, the invention relates to a production method for a surface-treated workpiece.

It is known in principle that an increase in the roughness of a metal workpiece surface can be helpful in improving the adhesion of outer layers. In particular, trenches, pores or local elevations can be structures on the workpiece surface which can be filled or surrounded by a flowable polymer, prepolymer or monomer, so that after it's curing the polymer film is fixed as a result of mechanical anchoring.

In medicine, the use of implants with porous surfaces for better fixation of the surrounding organic tissue has been known for more than 20 years.

Titanium and its alloys are a widely used material for implants in particular because of the high biocompatibility, good corrosion resistance and low toxicity.

The roughness of the implant made of titanium or its alloys necessary for successful osseointegration can be achieved by diffusion welding, sintering, sand blasting, plasma spraying, successive etching or other methods.

From Hacking et al. “Acid-etched microtexture for enhancement of bone growth into porous-coated implants” [Journal of Bone and Joint Surgery 85, 1182] it is known, that for forming a microtexture etching may be advantageous over sintering.

It is known from US 2007/0187253 A1 to remove beta (β) phases from Ti-6Al-4V alloys near the surface so as to produce a nanotopographic surface. The process is limited to the structuring of Ti-6Al-4V alloys. The described method is likewise limited to the removal of the β phase, if this is not formed, it can not be used, and further, other impurities and mechanical defects not given by the β phase remain on the surface and thus also do not contribute to favorable topography.

A further disadvantage of the process is that the structuring of the Ti-6Al-4V alloy surface requires the incorporation into an electrochemical cell with appropriate electrical contacting, which means that the contact point of the workpiece must not come into contact with the electrolyte, in order to ensure defined and reproducible electrochemical conditions during the process, and thus the entire workpiece can not be surface-modified.

It is an object of the invention to provide metallic workpieces which have a particularly favorable topography.

It is also an object of the invention to provide metallic workpieces with a favorable topography constituted of the metals titanium, zirconium, hafnium, vanadium, niobium, tantalum and the alloys of these metals as well as the nickel-titanium alloy known under the name “Nitinol”.

It is also an object of the invention to provide metallic workpieces which are free from inclusions and dispersions or precipitations of other metals and/or intermetallic phases as well as defect areas on their surface.

In addition, the object of the invention is to provide a method for patterning the surface, which can also be applied to workpieces made of titanium and/or titanium alloys of different compositions, as well as to other metals and alloys.

It is also an object of the invention to provide a process which, in addition to the β phase, also removes further impurities or mechanical defects from the surface and thus increases the purity of the surface.

Furthermore, it is an object of the invention to provide a method which makes it possible to structure complete workpieces, even with a complex shape.

The object of the invention is achieved by the provision of a metallic workpiece of the metals titanium, zirconium, hafnium, vanadium, niobium, tantalum and/or their alloys, as well as the nickel-titanium alloy known under the name “Nitinol”, of which the treated surface is largely free of inclusions and/or precipitations of other metals and/or intermetallic phases as well as mechanically strongly defect-rich regions (e.g., dislocation nests) and wherein the workpiece has a topography, which is defined by a first and a second roughness and wherein the first roughness is defined by depressions in the form of pores, and wherein the pores are open towards the surface and are closed towards the workpiece, and wherein at least 25%, preferably 50%, of the pores have an undercut, and wherein the second roughness is determined by statistically distributed elevations and depressions in the range of 100 nm and less.

Furthermore, the object of the invention is achieved by a method for producing the workpiece according to the invention which comprises the following steps

i. photochemical etching of the surface in an electrolyte under illumination
ii. chemical etching in acid solution

As workpieces the metals titanium, zirconium, hafnium, vanadium, niobium, tantalum and/or their alloys are suitable. Examples of suitable titanium alloys can be found in Materials Properties Handbook: Titanium Alloys, R. Boyer, G. Welsch, and E. W. Collings, eds. ASM International, Materials Park, Ohio, 1994. Particularly suitable among these are titanium grade 2, Ti-6Al-4V (grade 5) and derivatives of this alloy (grades 23-25, grade 29), Ti-3Al-2.5V (grade 9) and derivatives of these alloys (e.g., grade 18 and grade 28) as well as NiTi (Nitinol). In the case of niobium, suitable alloys would be e.g. C-103, C-129Y, C3009, Cb 752, FS85 and Nb1Zr.

In this case, it is generally advantageous to clean the workpiece of coarse soiling and grease/oil before the first etching step i. The cleaning is carried out in a manner which is generally known to a person of ordinary skill in the art, for example by rinsing with water-miscible organic solvents, e.g., by an acetone bath or a bath consisting of 70% isopropanol and 30% deionized water.

During the photochemical etching step i, a chemically greatest possible surface smoothness is produced. The electrolyte is preferably a strongly oxidizing acid.

Among oxidizing acids, those skilled in the art generally understand such acids which, in addition to the reaction potential resulting from the protons, still participate in a redox reaction. The anion of the oxidizing acid oxidizes the metal and is thereby reduced. The occurrence of the oxidizing effect here also depends on the concentration of the acid and the temperature in the manner known to the worker of ordinary skill. Typically, strongly oxidizing acids come from the group known as oxygen acids. The strong oxidizing acid is preferably selected from the group consisting of sulfuric acid (H₂SO₄), nitric acid (HNO₃), peroxomonosulfuric acid (H₂SO₅), chloric acid (HClO₃), perchloric acid (HClO₄), chromic acid (H₂CrO₄), arsenic acid (H₃AsO₄), hydrogen peroxide (H₂O₂) and/or a combination of these acids and/or one or a combination of these acids diluted with water (H₂O).

The illumination can be performed with LED arrays e.g. based on Enfis Uno Tag Arrays. The nominal irradiance (radiant flux) should be between 200 and 450 mW. The wavelengths can be in the range between 190 and 780 nm.

The shape of the sample container and the distance to the illumination source are to be selected in dependence on the sample geometry in such a way that homogeneous illumination is possible. Typically, a distance of 8 cm between sample surface and LED array was chosen.

The photochemical etching step i takes place at a temperature between 10° C. and 50° C., preferably between 15° C. and 40° C., particularly preferably between 20° C. and 25° C. (room temperature).

The duration of the photochemical etching step i is 5 to 30 hours, preferably 10 to 25 hours, more preferably 15 to 22 hours.

After photochemical etching, the sample is rinsed several times in a manner well known to those skilled in the art, e.g., with deionized water and subsequently dried e.g. by simple air drying before being placed in the etching bath ii.

The chemical etching step ii serves to remove inclusions and/or precipitations and/or mechanically defective areas and takes place in acidic solution. The acidic solution is a combination of a strongly oxidizing acid and an oxide-dissolving acid.

Acid-dissolving acids are understood as meaning those acids which are capable of converting metal oxides into metal salts with formation of water. Preferably, the oxide-dissolving acids are selected from the group consisting of hydrochloric acid (HCl), hydrofluoric acid (HF), bromic acid (HBr), iodic acid (HI), a combination of these acids and/or one or a combination of these acids in dilution with water (H₂O).

The etching solution for etching step ii (etching solution ii) can be selected from the group consisting of sulfuric acid (H₂SO₄), hydrochloric acid (HCl), hydrofluoric acid (HF), where appropriate, diluted with water.

The etching solution is typically based on a 1:1 mixture of concentrated sulfuric acid (96-98%) and concentrated hydrochloric acid (37%) or hydrofluoric acid (40%). Optionally, the ratio between concentrated sulfuric acid and concentrated hydrochloric acid/conc. hydrofluoric acid may be varied, e.g., to a ratio of 1:2. This etching solution ii can also be diluted by the addition of deionized water.

The chemical etching step ii is preferably carried out at room temperature. The sample is preferably added directly after the etching solution has been prepared. The etching bath is typically not stirred, since gas evolution occurs during etching, the gas evolution causing intermixing of the etching solution. The volume of the etching solution used is selected in such a way that the etching solution is present in excess in comparison to the sample volume and thus the sample volume is completely wetted. The etching step ii generally takes place at room temperature, without the introduction of external heat, but using the resulting heat of reaction.

The duration of the chemical etching step ii is between 1 and 6 hours and can be extended by diluting the etching bath. When the etching solution ii is undiluted, the duration of the etching step ii is preferably 2 to 4 hours, more preferably 3 hours.

After the chemical etching step ii, the surface-treated workpiece is repeatedly rinsed in a manner well known to the person of ordinary skill in the art, e.g. with deionized water and subsequently dried e.g. by simple air drying.

The workpieces according to the invention have a high purity and freedom of mechanical defects on the surface after the two-stage etching treatment.

Exemplary embodiments of the invention are described below with reference to the accompanying figures.

Therein:

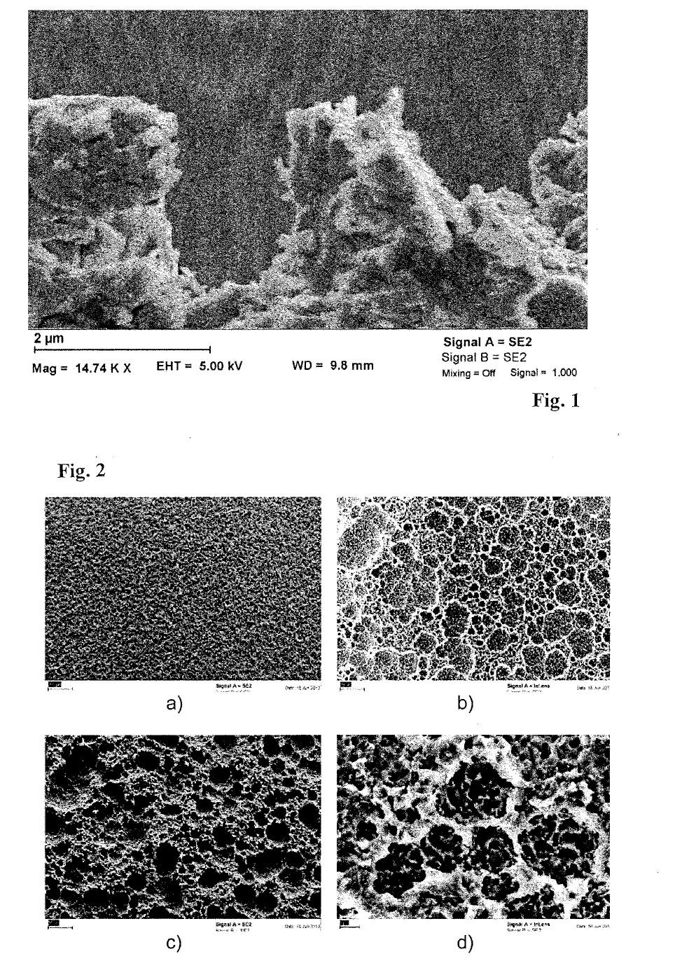

FIG. 1 shows a scanning electron microscopy, SEM, image of a workpiece according to the invention;

FIG. 2 scanning electron microscope top view of a Ti-3Al-2.5V surface treated according to the invention,
a) macroscopic overview,
b) microscopic survey,
c) same section and equal magnification, but secondary electron detector and
d) high magnification on the surface with cup-shaped indentations with undercut;

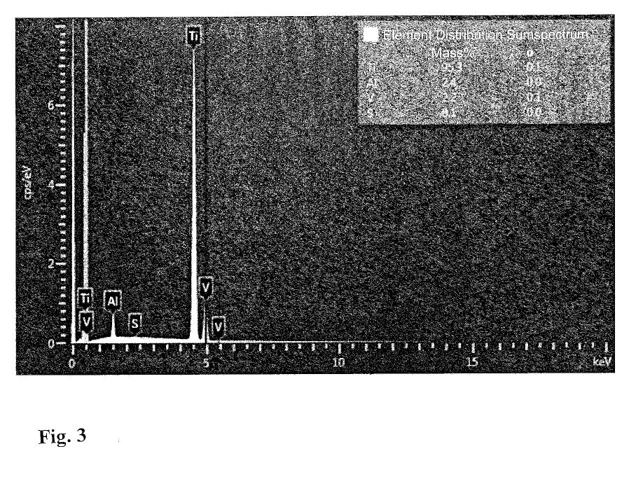

FIG. 3 EDX analysis at 15 keV: spectrum of the element distribution on the Ti-3Al-2.5V surface after processing according to the invention;

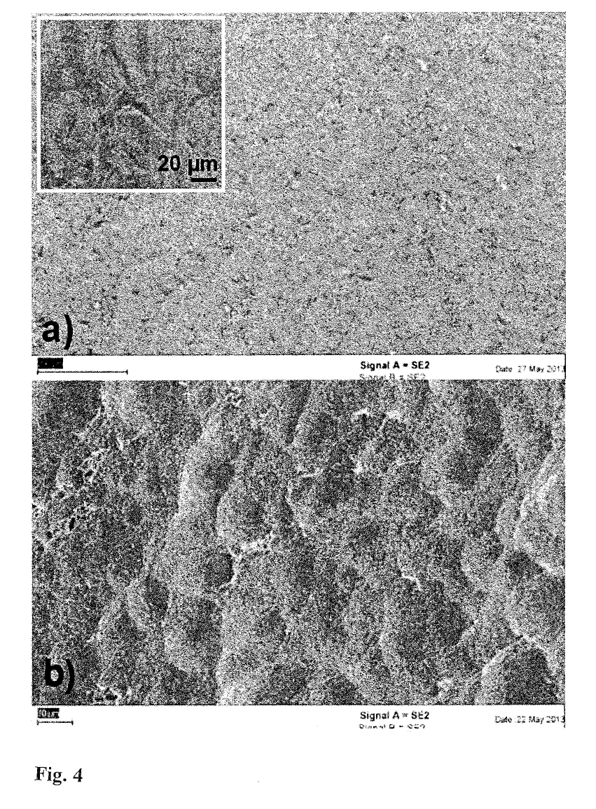

FIG. 4 scanning electron microscopy top view
a) after illumination and
b) after processing according to the invention, with different magnification;

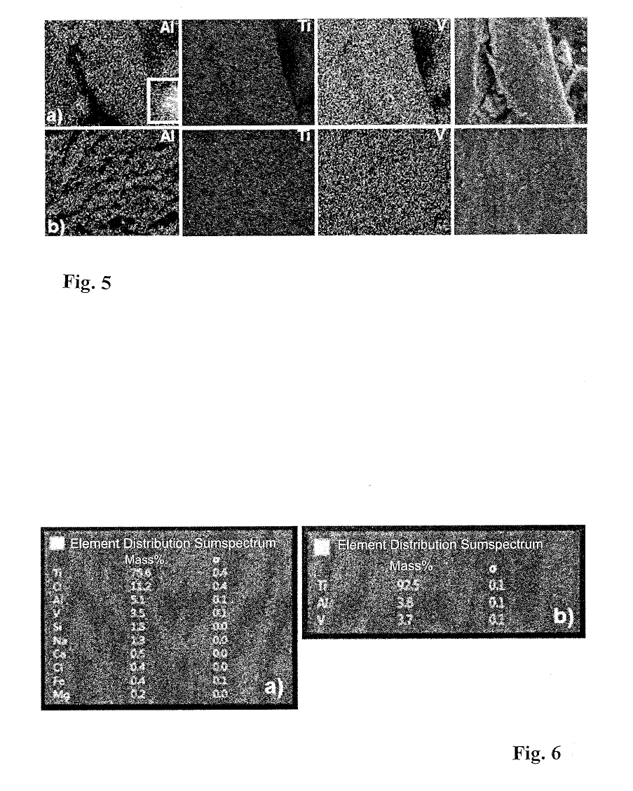

FIG. 5 EDX element distribution of Al, Ti and V as well as the corresponding surface,
a) after illumination and
b) after additional processing according to the invention;

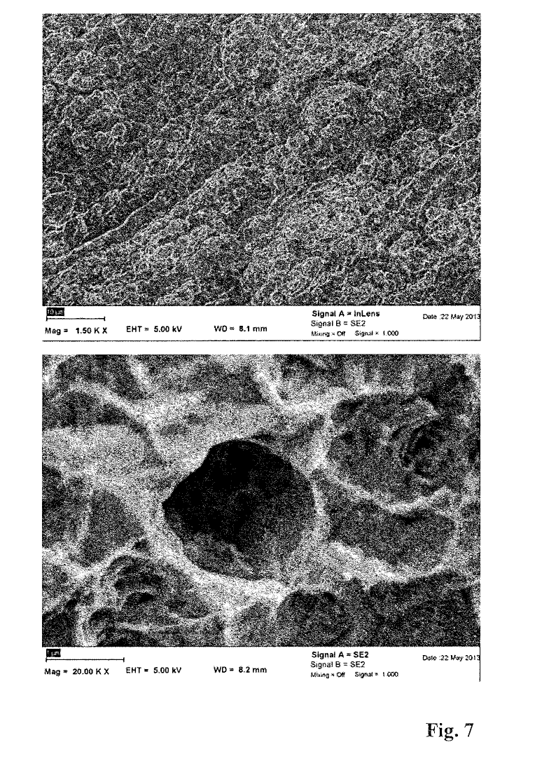

FIG. 6 EDX analysis at 15 keV: sum spectrum of the element distribution on the Ti-6Al-4V surface,
a) after irradiation/illumination and
b) after additional inventive processing;

FIG. 7 REM top view of the surface of a Ti-6Al-4V sample
a) after additional inventive processing (etching solution ii diluted with water)
b) in high magnification

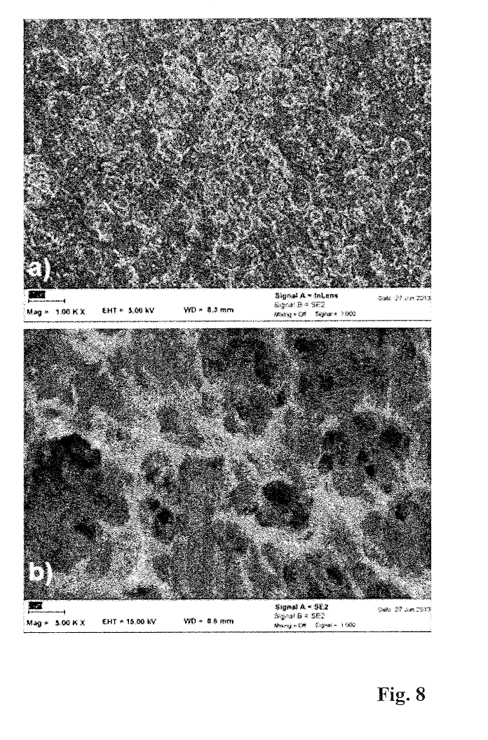

FIG. 8 REM top view of the surface of a Ti-6Al-4V sample
a) after additional inventive processing (etching solution i diluted with water)
b) in high magnification

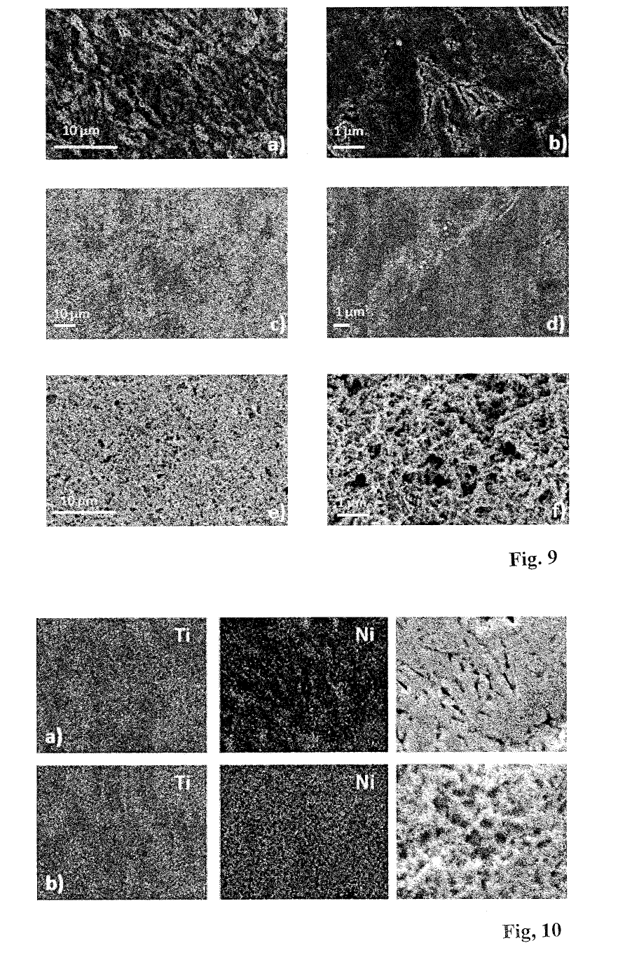

FIG. 9 REM top view of the surface of a NiTi alloy wire,
a) prior to processing,
b) in high magnification,
c) after the first etching step i,
d) in high magnification,
e) after complete processing according to the invention and
f) in high magnification;

FIG. 10 EDX element distribution of Ti and Ni as well as the corresponding surface,
a) untreated and
b) after complete processing according to the invention;

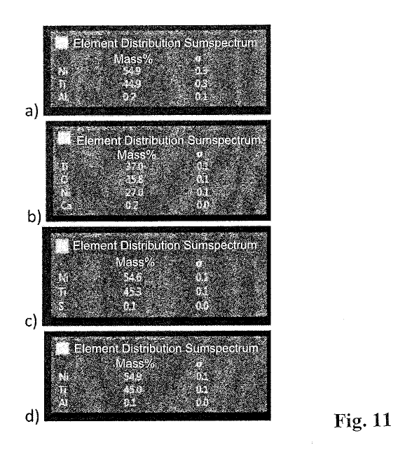

FIG. 11 EDX analysis at 15 keV: sum spectrum of the element distribution on the Ti-6Al-4V surface,
a) untreated in cross-section,
b) untreated on the surface,
c) after the first etching step i and
d) after complete processing according to the invention;

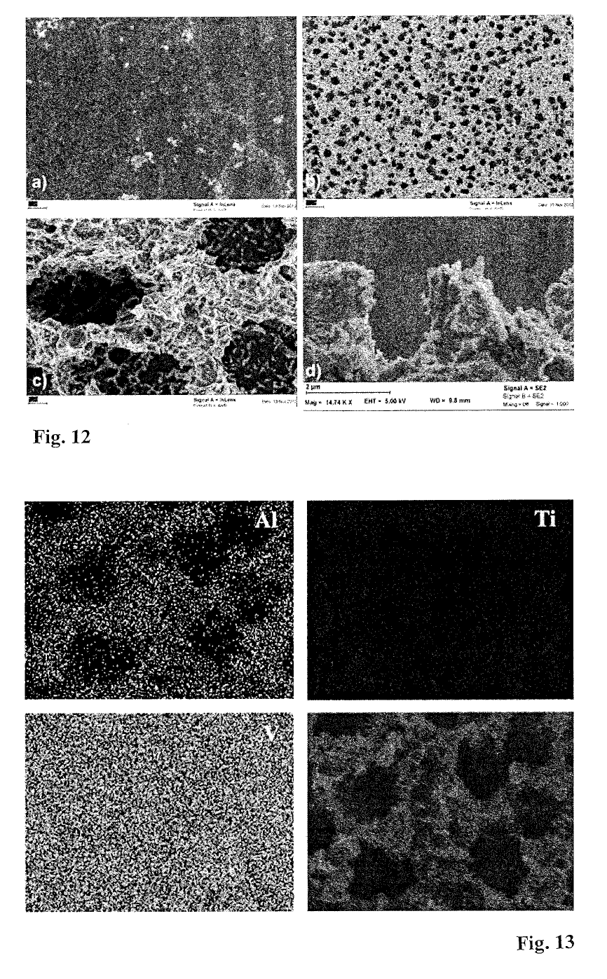

FIG. 12 REM Top view of the Ti grade 2 surface
a) etching step i for 20 h and
b) complete process according to the invention,
c) in high magnification and
d) in cross-section. At different magnifications;

FIG. 13 EDX analysis at 15 keV: element distribution of Ti, Al and V as well as the corresponding sample surface (Ti grade 2, 99.6% purity) after complete processing according to the invention.

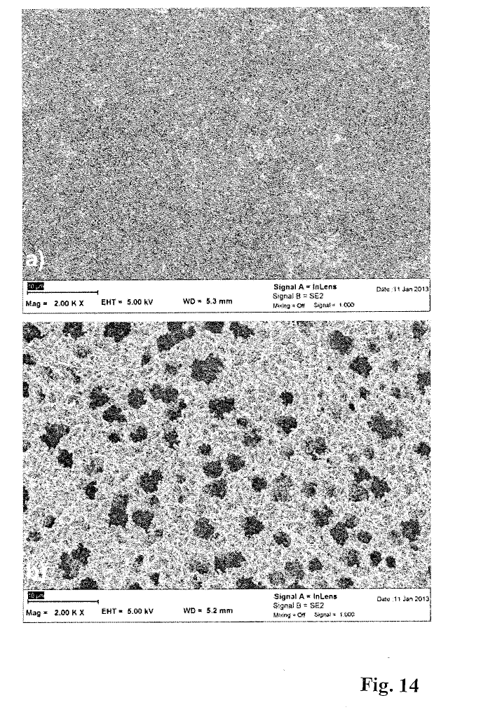

FIG. 14 REM Top view of the Ti grade 2 surface after
a) etching step i for 20 h (illumination in the UV range) and
b) complete inventive processing

FIG. 1 shows the image of a workpiece according to the invention, the image produced by means of scanning electron microscopy SEM. There can be seen clearly the first roughness in the form of pores, which are open towards the surface and closed towards the workpiece. A pore with a distinct undercut can be seen, which has a diameter which is larger than the diameter of the pore opening. Further, the second roughness is seen by statistically distributed elevations and depressions in the range of 100 nm and less.

Because of this topography comprised of a roughness in the nanoparticle and the pores with a diameter of 0.5 μm to 50 μm, preferably 1 μm to 40 μm, particularly preferably 2 μm to 20 μm with an undercut, the materials according to the invention are outstanding for the adhesive bonding with other materials. The workpieces can thus be used in the field of medical implant technology.

The workpieces according to the invention can be used e.g. for dental implants or artificial hip joints. For dental implants, the osseointegration of the Ti implant is of great importance for the long-term stability of the implant. The topography of pores with an undercut and the nanoroughness in the pores ensure excellent mechanical interlocking between bone and implant. With the Ti surface patterning, a purification of the Ti surface can also take place, which can result in a lower release of alloy metal ions from the implant, e.g. Al or V ions. This is especially significant e.g. for artificial hip and knee joints.

In the art, the workpieces according to the invention are particularly important in the field of composite materials, since the bonding of the various materials is important. Thus, the workpieces according to the invention can be used for composite turbine blades. The composite consists in this case of a layer sequence of fiber-reinforced polymer and Ti sheets, which are connected to one another. For this connection, optimum adhesion between polymer and Ti sheet is of the utmost importance. The pore undercuts and the nanoroughness of the pores provide the mechanical interlocking between the Ti sheet and the polymer.

In order not to limit the generality of the teaching, the invention will be explained in the following in some examples:

### EXAMPLE 1

### Ti-3Al-2.5V

### Manufacturing Process

The sample (turbine blade) was sandblasted and then cleaned for 5 min in acetone and dried in air. The sample is then etched for 24 h in concentrated H₂SO₄ at room temperature without external temperature control under illumination with an ENFIS Uno Day Red LED array operated at a nominal irradiance of 400 mW (300 mA and a wavelength of 620 nm) at a distance of approximately 8 cm from the sample surface. After this step, the sample was repeatedly cleaned in deionized water and dried in air. Subsequently, the sample was etched for 3 h in a freshly prepared fresh solution of HCl and H 2 SO 4 in a volume ratio of 1:1 without external temperature control. The sample was then repeatedly washed in deionized water and dried in air. The volumes of the respective etching solution were chosen such that a complete wetting of the sample is assured. The etching solutions were not stirred during the etching.

The scanning electron micrographs (SEM) in FIG. 2 show the microstructured surface of the sample Ti-3Al-2.5V (turbine blades) treated according to the invention. FIG. 2a shows that the surface is homogeneous without extreme differences in roughness. The magnification of the surface in FIG. 2b shows a cup-shaped structuring with differently sized cup diameters ranging from 2 μm to about 20 μm. Especially in the left part of FIG. 2b one sees the interpenetration of these cups, which leads to even larger cups. This interpenetration is best seen in FIG. 2c. FIG. 2d shows the surface in high magnification. It can be seen that the cup-like depressions have small sharp-edged sub-microstructures. The lighter/white edges around the cup-like recesses are caused by the undercuts that occur during the etching process. The undercut structure together with the small sharp-edged sub-microstructures in and between the cup-like recesses allow for a significant improvement in the mechanical interlock between the titanium substrate and a layer applied thereupon consisting e.g. of plastic. The undercuts, as well as the sub-microstructures, act as a barb.

FIG. 3 shows the spectrum of the element distribution on the Ti-3Al-2.5V surface. It shows that, after processing, the aluminum content of the alloy near the surface has been reduced by about 20%, while the decrease in the vanadium content is substantially less. The sulfur signal is presumably an artifact due to the insufficient thorough rinsing of the sample after the etching process.

### EXAMPLE 2

### Ti-6Al-4V

### Manufacturing Process

The sample was degreased for 5 min in acetone and cleaned and air-dried. The sample is then etched for 20 h in concentrated H₂SO₄ at room temperature without external temperature control under illumination with an ENFIS Uno Tag Red LED array operated at a nominal irradiance of 400 mW (300 mA and a wavelength of 620 nm) at a distance of approximately 8 cm from the sample surface. After this step, the sample was repeatedly cleaned in deionized water and dried in air. Subsequently, the sample was etched for 3 h in freshly prepared etching solution consisting of HCl and H₂SO₄ in a volume ratio of 1:2 without external temperature control. The sample was then repeatedly washed in deionized water and dried in air. The volumes of the respective etching solution were chosen such that a complete wetting of the sample is assured. The etching solutions were not stirred during the etching.

The SEM images are shown in FIG. 4. FIG. 4a shows the sample before the treatment according to the invention, a blasted Ti-6Al-4V surface can be seen. One can see many nicks and deep scratches with a length of typically about 20 μm—see section on the upper right of FIG. 4a. These surface defects can function as nucleation nuclei for cracks, which can lead in the worst case to failure of the workpiece. FIG. 4b shows the Ti-6Al-4V surface after treatment according to the invention. The surface shows a weak-cup-like surface with a smooth nanostructure. The surface defects clearly visible in FIG. 4a were completely removed by processing.

FIG. 5 shows the EDX element mapping of Ti, Al and V of the two Ti-6Al-4V surfaces shown in FIG. 4. FIG. 5a shows very rich Al-rich regions in the white-framed region, most probably as grains of intermetallic phases. These grains can also function as nucleation nuclei for cracks, in addition to the ridges and scratches shown in FIG. 4a. According to the invention, no Al-rich regions are found on the surface.

The integral superficial element distribution of the blasted sample (FIG. 6a) shows comparatively high proportions of alkali metals as well as Fe and Si. These may be e.g. residues of the blasting process. After processing according to the invention, all residues of alkali metals as well as Fe and Si are removed. Compared to the surface not processed according to the invention, the concentration of Al is very greatly reduced, the V content remaining constant.

### EXAMPLE 3

### Ti-6Al-4V (Etching Solution ii with Water Component)

### Manufacturing Process

The sample is cleaned for 5 min in acetone and degreased and dried in air. The sample is then etched for 20 h in concentrated H₂SO₄ at room temperature without external temperature control under illumination with an ENFIS Uno Day Red LED array operated at 300 mA (400 mW) at a distance of approximately 8 cm from the sample surface. After this step, the sample was repeatedly cleaned in deionized water and dried in air. Subsequently, the sample is etched for 3 h in freshly prepared etching solution consisting of HCl, H₂SO₄, H₂O (deionized) in a volume ratio of 1:1:1 without external temperature control. The sample was then repeatedly washed in deionized water and dried in air. The volumes of the respective etching solutions were chosen such that a complete wetting of the sample is assured. The etching solutions were not stirred during the etching.

FIG. 7 a) shows the Ti-6Al-4V surface after the two-step etching process. One can see a fine-formed cup-like surface with a rough nanostructure. The diameters of the cups are about 2 μm. In part, these structures have also grown into one another. FIG. 7 b) shows such a cup-like structure with its nanostructured walls. In contrast to FIG. 4 b), however, the cup-like structures are substantially smaller in diameter and much flatter. In addition, they have a lighter undercut, as can be seen in FIG. 7 b). The surface defects seen in FIG. 4 a) were completely removed by processing.

### EXAMPLE 4

### Ti-6Al-4V (Etching Solution i with Water Component)

### Manufacturing Process

The sample is cleaned for 5 min in acetone and dried in air. Subsequently, the sample is etched for 20 h in an etching solution consisting of concentrated H₂SO₄ and deionized water (volume ratio 1:1) at room temperature without external temperature control under illumination with an ENFIS Uno Tag Red LED array operated at 300 mA (400 mW) about 8 cm from the sample surface. After this step, the sample was repeatedly cleaned in deionized water and dried in air. The sample is then washed for 3 h in a freshly prepared etching solution consisting of cone. HCl and conc. H₂SO₄ in the volume ratio 1:1 without external temperature control. The sample was then repeatedly washed in deionized water and dried in air. The volumes of the respective etching solution were chosen such that a complete wetting of the sample is assured. The etching solutions were not stirred during the etching.

FIG. 8 a) shows the Ti-6Al-4V surface after the two-stage etching process. A finely formed cup-like surface with a rougher nanostructure than in FIG. 7 a) can be seen. The diameters of the cups are about 4 μm. In part, these structures have also grown into one another. FIG. 8 b) shows such a cup-like structure consisting of several smaller cups and nanostructured walls. In contrast to FIG. 7 b), however, the cup-like structures are substantially smaller in diameter and much flatter. In addition, they have a slight undercut, as can be seen in FIG. 8 b). The surface defects seen in FIG. 4 a) were completely removed by the processing.

### EXAMPLE 5

### NiTi

### Manufacturing Process

The sample was degreased for 5 min in acetone and cleaned and air-dried. Subsequently, the sample is etched for 20 h in concentrated H₂SO₄ at room temperature without external temperature control under illumination with an ENFIS Uno Day Red LED array at an irradiation strength of 200 mW (200 mA) at a distance of approximately 8 cm from the sample surface. Since the NiTi wire should be structured from all sides at the same time, the NiTi wire was rotated around its own axis at about 10 rpm with stationary, one-sided illumination. After this step, the sample was repeatedly cleaned in deionized water and dried in air. The sample was then etched for 2 h in freshly prepared etching solution consisting of HCl and H₂SO₄ in a volume ratio of 1:2 without external temperature control. The sample was then repeatedly washed in deionized water and dried in air. The volumes of the respective etching solution were chosen such that a complete wetting of the sample is assured. The etching solutions were not stirred during the etching.

The SEM images in FIG. 9 show the surface of wires made of a NiTi alloy. FIG. 9 a and b show the untreated surface. It is traversed by many deep but narrow furrows whose shape is irregular and usually elongated. The origin of these furrows is therefore presumably in the process of wire drawing. FIG. 9 c and d show the surface structure after the first etching step i. The surface has no furrows and is much smoother compared to the untreated surface. In places with deep furrows, these are almost completely leveled. FIG. 9 e and f show the completely changed surface according to the invention. It now has many sharp edges and corners, as well as cup-like depressions, which consist of several “holes” provided with undercuts. Some of these “holes” have grown together. The surface appears homogeneous with respect to the set roughness.

FIG. 10 shows the elemental distribution of Ti, Ni on the additionally developed sample surface. The untreated sample shows a structural dependence between the nickel concentration on the surface and the surface structure (FIG. 10a). This also applies to a lesser extent for Ti. This relationship between structure and Ni/Ti concentration is no longer present after the process according to the invention. The surface is homogeneous with respect to the Ni and Ti distribution.

The sum spectrum of the treated and untreated sample is shown in FIG. 11. In the volume of the NiTi wire, the concentration of Ni is significantly higher than that of Ti (FIG. 11a). In addition, there is a low concentration of Al. This relationship is reversed at the surface. There is more Ti than Ni (FIG. 11b). In addition, the surface is strongly oxidized and there is Ca on the surface. After the first etching step (FIG. 11c), the ratio between Ni and Ti on the surface changes again and approaches the bulk values. After complete processing according to the invention, almost no difference between the bulk and the surface can be observed (FIG. 11d).

### EXAMPLE 6

### Ti (Grade 2)

### Manufacturing Process

The sample was burnished with abrasive paper (4000 SiC grain size) until the cutting or saw marks were removed and then degreased for 5 min in acetone and cleaned and dried in air. The sample is then etched for 20 h in concentrated H₂SO₄ at room temperature without external temperature control under illumination with an ENFIS Uno Tag Red LED array operated at a nominal irradiance of 400 mW (300 mA and a wavelength of 620 nm) at a distance of approximately 8 cm from the sample surface. After this step, the sample was repeatedly cleaned in deionized water and dried in air. Subsequently, the sample is etched for 3 h in freshly prepared etching solution consisting of HCl and H₂SO₄ in a volume ratio of 1:1 without external temperature control. The sample was then repeatedly washed in deionized water and dried in air. The volumes of the respective etching solution were chosen such that a complete wetting of the sample is given. The etching solutions were not stirred during the etching.

FIG. 12 shows the surface of Ti (grade 2) after the individual etching steps. 12 a) shows a smooth surface after the photochemical etching step i. The bright spots are the deposition of salts on the surface due to insufficient cleaning. The surface resulting after complete processing according to the invention is shown in FIG. 12 b. The surface has a high density of pores. Partially, they begin to grow together. Their basic shape is rounded/oval with irregularly shaped edges. FIG. 12 c shows that these pores have sharp-edged sub-microstructures on the wall and at the tip of the pore. This is also shown in the cross-section in FIG. 12 d. These pores typically have an undercut. This is advantageous, together with the sharp-edged sub-microstructures on the pore wall, for the mechanical interlocking with a layer applied to the surface, e.g. a polymer.

The EDX element distribution in FIG. 13 shows that there is a local depletion of Al in the region of the pores, whereas the Ti and V concentrations are unaffected. When e.g. intermetallic phases such as Ti₃Al are present on the surface, these are selectively etched out compared to the host matrix (Ti plus impurities as a mixed crystal). As a result, the pores are formed on the surface.

### EXAMPLE 7

### Ti (grade 2) Illumination During the Etching Step i in the UV Range

### Manufacturing Process

The sample was burnished with abrasive paper (4000 SiC grain size) until the cutting or saw marks were removed and then cleaned for 5 min in acetone and dried in air. Subsequently, the sample is etched for 24 h in concentrated H₂SO₄ at room temperature without external temperature control under illumination with an ENFIS Uno Day UV LED array at 300 mA at a distance of approximately 8 cm from the sample surface. After this step, the sample was repeatedly cleaned in deionized water and dried in air. Subsequently, the sample was etched for 3 h in a freshly prepared fresh solution of HCl and H₂SO₄ in a volume ratio of 1:1 without external temperature control. The sample was then repeatedly washed in deionized water and dried in air. The volumes of the respective etching solution were chosen such that a complete wetting of the sample is assured. The etching solutions were not stirred during the etching.

FIG. 14 shows the surface of Ti grade 2 after the individual etching steps. FIG. 14 a shows a smooth surface after the etching step i. The bright spots are deposits of salts on the surface due to insufficient cleaning. The surface obtained after complete processing is shown in FIG. 14 b. The surface has a high density of pores. They partially begin to grow together. Compared to FIG. 12b, the pores are more jagged in their shape.

1. A surface-treated metallic workpiece of titanium and/or titanium alloys with titanium as the main component and/or nickel-titanium alloys, as well as Nitinol, wherein the metal on the treated surface is free of inclusions, precipitations of other metals, deposits of alkali, alkaline earth metals and/or aluminum, intermetallic phases, and/or areas which are mechanically highly defective, and wherein the surface has a first roughness and a second roughness, in which the first roughness is given by depressions in the form of pores, wherein the pores
have a diameter in the range between 0.5 and 50 μm
are open towards the surface
and
are closed in the direction of the workpiece,
and at least a part of the pores have an undercut section
and the second roughness is given by statistically distributed elevations and depressions in the range of 100 nm and less.

2. A method for production of the surface-treated metallic workpiece according to claim 1, comprising successively:
i. photochemical etching the metallic workpiece in the presence of an acidic electrolyte with simultaneous illuminating, the acid electrolyte being a strong oxidizing acid
and
ii. chemical etching the product of step i. in an acidic solution, the acidic solution containing a combination of a strongly oxidizing acid and an oxide-dissolving acid.

3. The method according to claim 2, wherein the illuminating is carried out at an illumination intensity of 200 to 450 mW and a wavelength between 190 and 780 nm.

4. The method according to claim 2, wherein between the step i.-etching and the step ii.-etching or after the step i.-etching and the step ii.-etching, rinsing and drying takes place.

5. The method according to claim 2, wherein the strongly oxidizing acid is selected from the group of oxygen acids (oxyacids).

6. The method according to claim 2, wherein the strongly oxidizing acid is at least one of (H₂O₅), chloric acid (HClO₃), perchloric acid (HClO₄), chromic acid (H₂CrO₄), arsenic acid (H₃AsO₄) and hydrogen peroxide (H₂O₂).

7. The method according to claim 2, wherein the oxide-dissolving acid is at least one of (HCl), hydrofluoric acid (HF), bromic acid (HBr) and iodic acid (HI).

8. The method according to claim 5, wherein the strongly oxidizing acid or the oxide-dissolving acid is diluted with water.
